(19) United States
(12) Patent Application Publication
(10) Pub. No.: US 2026/0297009 A1
(43) Pub. Date: Oct. 1, 2026

(54) PROCESSES FOR THE PRODUCTION OF LACTIC ACID WITH CONCURRENT RECOVERY AND REUSE OF BY-PRODUCTS THEREOF

(71) Applicant: TRIPLEW LTD., Netanya (IL)

(72) Inventors: Tal Shapira, Brasschaat (BE); Maarten Campman, Roosendaal (NL); Nitsan Papo, Hod Hasharon (IL)

(21) Appl. No.: 19/480,464

(22) PCT Filed: May 2, 2024
(86) PCT No.: PCT/IL2024/050421

§ 371 Date: Oct. 31, 2025

Related U.S. Application Data
(60) Provisional application No. 63/499,719, filed on May 3, 2023.
(60) Provisional application No. 63/510,368, filed on Jun. 27, 2023.
(60) Provisional application No. 63/515,602, filed on Jul. 26, 2023.

(51) Int. Cl. C07C 51/02; C01F 5/00

(52) U.S. Cl. CPC C07C 51/02; C01F 5/00

(57) ABSTRACT

Economically and environmentally beneficial processes for the preparation of lactic acid are provided. The processes produce lactic acid with concurrent recovery and reuse of residues formed in the processes thereby being essentially free of undesired by-products.

### FIELD OF THE INVENTION

The present invention relates to processes for preparing lactic acid with concurrent recovery and recycling of residues and by-products formed therein.

### BACKGROUND OF THE INVENTION

Lactic acid is the most widely occurring hydroxycarboxylic acid with applications in food, chemical, pharmaceutical, and cosmetic industries. This naturally occurring organic acid can be produced by chemical synthesis or microbial fermentation. When produced by microbial fermentation, care should be taken to avoid endogenous decrease in pH due to the formation of lactic acid in order to maintain the productivity of the microorganisms. A pH in the range of 5-7 is preferable and can be obtained by the addition of bases such as monovalent or divalent hydroxides that neutralize the lactic acid thereby producing a lactate salt with the corresponding monovalent or divalent cations. In order to convert the lactate salt to lactic acid, an acidification step needs to be performed.

GB 173,479 describes a process of purifying lactic acid, characterized by lactic acid, or its salts, being first converted into magnesium lactate which (if necessary after purification) is decomposed with acid and extracted at ordinary temperature or with heat in a suitable solvent, such as ether, acetone or the like, after the distillation of which pure lactic acid remains.

WO 2000/017378 describes a process for the preparation of lactic acid, comprising a fermentation reaction carried out at a pH in the range of between 5.5 to 6.5, wherein the pH is adjusted to said range by introducing into the reaction mixture M(OH)₂, wherein M is Ca or Mg, to obtain M-lactate-containing broth and recovering the lactic acid from said M-lactate by reacting the same with HCl, either by treating the M-lactate-containing broth with HCl or by precipitating M-lactate from the broth, and subsequently reacting said precipitate with HCl, to yield a lactic acid solution, and extracting and/or purifying lactic acid from said solution.

WO 2005/123647 describes a process for the preparation of lactic acid and/or lactate from a medium comprising magnesium lactate, wherein the magnesium lactate is reacted with a hydroxide of sodium, potassium, calcium, and/or ammonium at a pH range between 9 and 12, preferably between 9.5 and 11, to form a lactate of sodium, potassium, calcium and/or ammonia and magnesium hydroxide.

WO 2011/095631 describes a process for the preparation of lactic acid comprising the steps of: a) providing an aqueous medium comprising magnesium lactate; b) adding to the aqueous medium comprising magnesium lactate a monovalent base to form an aqueous medium comprising a water soluble monovalent lactate salt and a solid magnesium base; c) separating the magnesium base from the aqueous medium comprising the water soluble monovalent lactate salt; d) adjusting the concentration of the monovalent lactate salt in the aqueous medium to a value between 10 and 30 wt. %; e) subjecting the aqueous medium comprising the monovalent lactate salt to water-splitting electrodialysis, to produce a first solution comprising monovalent base and a second solution comprising lactic acid and monovalent lactate salt, the electrodialysis being carried out to a partial conversion of 40 to 98 mole %; f) separating the second solution comprising lactic acid and monovalent lactate salt into lactic acid and a solution comprising the monovalent lactate salt by vapour-liquid separation; g) recycling the solution of step f) comprising the monovalent lactate salt to step d).

WO 2023/006876 describes a process for the preparation of lactic acid comprising the steps of: a) providing an aqueous medium comprising magnesium lactate; b) adding to the aqueous medium comprising magnesium lactate a monovalent base to form an aqueous medium comprising a water soluble monovalent lactate salt and a solid magnesium base; c) separating the solid magnesium base from the aqueous medium comprising the water soluble monovalent lactate salt; d) providing an aqueous medium comprising the water soluble monovalent lactate salt at a concentration of more than 30 wt. % and at most 45 wt. %; e) subjecting the aqueous medium comprising the water soluble monovalent lactate salt from step d) to water-splitting electrodialysis, to produce a first solution comprising monovalent base and a second solution comprising lactic acid and monovalent lactate salt, the electrodialysis being carried out to a partial conversion of 40 to 99 mole %; f) recovering lactic acid from the second solution comprising lactic acid and monovalent lactate salt.

There remains an unmet need for eco-friendly and cost-effective processes for the preparation of lactic acid.

### SUMMARY OF THE INVENTION

The present invention provides processes for preparing high-purity lactic acid with minimal by-products and residues. In some embodiments, the present invention provides a process which comprises the acidification of a lactate salt containing divalent cations using an acid thereby obtaining lactic acid and a salt residue containing the divalent cations, reacting the salt residue containing the divalent cations with a monovalent base thereby obtaining a divalent base and a salt residue containing the monovalent cations, and subjecting the salt residue containing the monovalent cations to subsequent use or to electrodialysis thereby obtaining a monovalent base and an acid. In other embodiments, the present invention provides a process which comprises reacting a lactate salt containing monovalent cations with a salt containing divalent cations thereby obtaining a lactate salt containing the divalent cations and a salt residue containing the monovalent cations, subjecting the salt residue containing the monovalent cations to subsequent use or to electrodialysis thereby obtaining a monovalent base and an acid, and acidification of a lactate salt containing divalent cations using an acid thereby obtaining lactic acid and a salt residue containing the divalent cations. In yet other embodiments, the present invention provides a process which comprises reacting a lactate salt containing divalent cations with a monovalent base thereby obtaining a divalent base and a lactate salt containing the monovalent cations, acidification of the lactate salt containing monovalent cations using an acid thereby obtaining lactic acid and a salt residue containing the monovalent cations, and subjecting the salt residue containing the monovalent cations to subsequent use or to electrodialysis thereby obtaining a monovalent base and an acid. In further embodiments, the present invention provides a process which comprises acidification of a lactate salt containing monovalent cations using an acid thereby obtaining lactic acid and a salt residue containing the monovalent cations, and subjecting the salt residue containing the monovalent cations to subsequent use or to electrodialysis thereby obtaining a monovalent base and an acid.

According to the principles of the present invention, the by-products of lactic acid formation obtained by the processes of the present invention are recovered and recycled, preferably as reagents in the various steps of the processes therefore providing economically and environmentally beneficial processes which are essentially free of undesired residues and by-products.

The present invention is based, in part, on the unexpected finding of by-product free processes for preparing lactic acid from a lactate salt. Contrary to hitherto known processes of acidulation of a lactate salt which typically produce large quantities of residual by-products, the processes of the present invention afford nearly zero by-products as they utilize multiple steps that are designed to recycle the by-products, for example as reagents that can be reused in the processes. Furthermore, the processes disclosed herein directly produce lactic acid in high purity and enhanced yield.

According to a first aspect, the present invention provides a process for the preparation of lactic acid, the process comprising the steps of:
a. providing an aqueous medium comprising a lactate salt with divalent cations;
b. adding an acid to the aqueous medium of step (a) to obtain lactic acid and a salt residue comprising the divalent cations;
c. reacting the salt residue comprising the divalent cations of step (b) with a first base comprising monovalent cations to obtain a second base comprising the divalent cations and a salt residue comprising the monovalent cations; and
d. collecting the salt residue comprising the monovalent cations of step (c),
wherein the second base comprising the divalent cations of step (c) is recovered and reused in the process of producing an aqueous medium of step (a).

According to certain embodiments, the divalent cations are magnesium ions. According to other embodiments, the divalent cations are calcium ions.

According to additional embodiments, the acid in step (b) is selected from the group consisting of sulfuric acid, hydrochloric acid, hydrobromic acid, nitric acid, carbonic acid, phosphoric acid, and combinations thereof. Each possibility represents a separate embodiment. In one embodiment, the acid in step (b) is sulfuric acid.

According to several embodiments, the salt residue comprising the divalent cations is selected from the group consisting of MgCl₂·xH₂O where x=0-6; MgCO₃·xH₂O where x=0, 1, 2, 3, or 5; MgSO₄·xH₂O where x=0-11; Mg₃(PO₄)₂·xH₂O where x=0, 5, 8, or 22; MgHPO₄·xH₂O where x=0 or 3; and Mg(H₂PO₄)₂·xH₂O where x=0, 2 or 4. Each possibility represents a separate embodiment. In one embodiment, the salt residue comprising the divalent cations is magnesium sulfate (e.g., magnesium sulfate heptahydrate).

According to some embodiments, the lactic acid obtained in step (b) is recovered using an organic solvent. In various embodiments, the organic solvent is selected from the group consisting of a ketone, an ether, an aldehyde, and a mixture thereof. Each possibility represents a separate embodiment. In one embodiment, the lactic acid obtained in step (b) is recovered using acetone. In certain embodiments, the salt residue comprising the divalent cations obtained in step (b) is recovered using an organic solvent before being used in step (c). In various embodiments, the organic solvent is selected from the group consisting of a ketone, an ether, an aldehyde, and a mixture thereof. Each possibility represents a separate embodiment. In one embodiment, the salt residue comprising the divalent cations obtained in step (b) is recovered using acetone. In currently preferred embodiments, the organic solvent is at a temperature of about 5° C. to about 25° C., including each value within the specified range.

According to certain embodiments, the first base comprising monovalent cations in step (c) is selected from the group consisting of sodium hydroxide, potassium hydroxide, ammonium hydroxide, and a mixture thereof. Each possibility represents a separate embodiment. According to other embodiments, the second base comprising the divalent cations in step (c) is magnesium hydroxide. According to yet other embodiments, the salt residue comprising the monovalent cations is selected from the group consisting of sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof. Each possibility represents a separate embodiment. In one embodiment, the salt residue comprising the monovalent cations is sodium sulfate. In another embodiment, the salt residue comprising the monovalent cations is ammonium sulfate.

According to certain embodiments, the process further comprises step (e) of subjecting the collected salt residue comprising the monovalent cations of step (d) to electrodialysis thereby obtaining a base comprising the monovalent cations and an acid, wherein the base comprising the monovalent cations is recovered and reused as the first base in step (c), and/or the acid is recovered and reused in step (b).

According to a second aspect, the present invention provides a process for the preparation of lactic acid, the process comprising the steps of:
a. providing an aqueous medium comprising a lactate salt with monovalent cations;
b. reacting the lactate salt with monovalent cations of step (a) with a first salt comprising divalent cations to obtain a lactate salt with divalent cations and a second salt residue comprising the monovalent cations;
c. adding an acid to the lactate salt with divalent cations of step (b) to obtain lactic acid and a salt residue comprising the divalent cations; and
d. collecting the second salt residue comprising the monovalent cations of step (b),
wherein the salt residue comprising the divalent cations of step (c) is recovered and reused as the first salt in step (b).

According to certain embodiments, the monovalent cations are sodium ions. According to other embodiments, the monovalent cations are ammonium ions.

According to additional embodiments, the first salt comprising divalent cations is selected from the group consisting of MgCl₂·xH₂O where x=0-6; MgCO₃·xH₂O where x=0, 1, 2, 3, or 5; MgSO₄·xH₂O where x=0-11; Mg₃(PO₄)₂·xH₂O where x=0, 5, 8, or 22; MgHPO₄·xH₂O where x=0 or 3; and Mg(H₂PO₄)₂·xH₂O where x=0, 2 or 4. Each possibility represents a separate embodiment. In one embodiment, the first salt comprising the divalent cations is magnesium sulfate (e.g., magnesium sulfate heptahydrate).

According to further embodiments, the acid in step (c) is selected from the group consisting of sulfuric acid, hydrochloric acid, hydrobromic acid, nitric acid, carbonic acid, phosphoric acid, and combinations thereof. Each possibility represents a separate embodiment. In one embodiment, the acid in step (c) is sulfuric acid.

According to some embodiments, the lactic acid obtained in step (c) is recovered using an organic solvent as detailed above.

According to other embodiments, the salt residue comprising the divalent cations of step (c) is recovered using an organic solvent as detailed above before being reused in step (b).

According to yet other embodiments, the second salt residue comprising the monovalent cations is selected from the group consisting of sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof. Each possibility represents a separate embodiment. In one embodiment, the salt residue comprising the monovalent cations is sodium sulfate. In another embodiment, the salt residue comprising the monovalent cations is ammonium sulfate.

According to further embodiments, the process further comprises step (e) of subjecting the collected second salt residue comprising the monovalent cations of step (d) to electrodialysis thereby obtaining a base comprising the monovalent cations and an acid, wherein the base comprising the monovalent cations is recovered and reused in the process of producing an aqueous medium of step (a), and/or the acid is recovered and reused in step (c).

According to another aspect, the present invention provides a process for the preparation of lactic acid, the process comprising the steps of:
a. providing an aqueous medium comprising a lactate salt with divalent cations;
b. reacting the lactate salt with divalent cations of step (a) with a first base comprising monovalent cations to obtain a second base comprising the divalent cations and a lactate salt comprising the monovalent cations;
c. adding an acid to the lactate salt comprising the monovalent cations of step (b) to obtain lactic acid and a salt residue comprising the monovalent cations; and
d. performing electrodialysis on the salt residue comprising the monovalent cations of step (c) to obtain a base comprising the monovalent cations and an acid,
wherein the second base comprising the divalent cations of step (b) is recovered and reused in the process of producing an aqueous medium of step (a), the base comprising the monovalent cations of step (d) is recovered and reused as the first base in step (b), and/or the acid in step (d) is recovered and reused in step (c).

According to some embodiments, the divalent cations are magnesium ions. According to other embodiments, the divalent cations are calcium ions.

According to certain embodiments, the first base comprising monovalent cations in step (b) is selected from the group consisting of sodium hydroxide, potassium hydroxide, ammonium hydroxide, and a mixture thereof. Each possibility represents a separate embodiment.

According to other embodiments, the second base comprising the divalent cations in step (b) is magnesium hydroxide.

According to further embodiments, the acid in step (c) is selected from the group consisting of sulfuric acid, hydrochloric acid, hydrobromic acid, nitric acid, carbonic acid, phosphoric acid, and combinations thereof. Each possibility represents a separate embodiment. In one embodiment, the acid in step (c) is sulfuric acid.

According to other embodiments, the lactic acid obtained in step (c) is recovered using an organic solvent as detailed above.

According to yet other embodiments, the salt residue comprising the monovalent cations obtained in step (c) is selected from the group consisting of sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof. Each possibility represents a separate embodiment. In one embodiment, the salt residue comprising the monovalent cations is sodium sulfate.

According to yet another aspect, the present invention provides a process for the preparation of lactic acid, the process comprising the steps of:
a. providing an aqueous medium comprising a lactate salt with monovalent cations;
b. adding an acid to the lactate salt with monovalent cations of step (a) to obtain lactic acid and a salt residue comprising the monovalent cations; and
c. performing electrodialysis on the salt residue comprising the monovalent cations of step (b) to obtain a base comprising the monovalent cations and an acid,
wherein the base comprising the monovalent cations of step (c) is recovered and reused in the process of producing an aqueous medium of step (a), and/or the acid in step (c) is recovered and reused in step (b).

According to additional embodiments, the acid for the acidulation of the lactate salt with monovalent cations to obtain lactic acid is selected from the group consisting of sulfuric acid, hydrochloric acid, hydrobromic acid, nitric acid, carbonic acid, phosphoric acid, and combinations thereof. Each possibility represents a separate embodiment. In one embodiment, the acid for the acidulation of the lactate salt with monovalent cations to obtain lactic acid is sulfuric acid.

According to some embodiments, the lactic acid obtained is recovered using an organic solvent as detailed above.

According to certain embodiments, the salt residue comprising the monovalent cations is selected from the group consisting of sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof. Each possibility represents a separate embodiment. In one embodiment, the salt residue comprising the monovalent cations is sodium sulfate.

According to certain embodiments, the electrodialysis on the salt residue comprising the monovalent cations in the processes disclosed therein is bipolar membrane electrodialysis (BPED). In some embodiments, the electrodialysis is electro-electrodialysis (EED). In additional embodiments, the electrodialysis is water-splitting electrodialysis. In other embodiments, the base obtained in the electrodialysis is selected from sodium hydroxide, potassium hydroxide, and ammonium hydroxide. Each possibility represents a separate embodiment. In yet other embodiments, the acid obtained in electrodialysis is selected from the group consisting of sulfuric acid, hydrochloric acid, hydrobromic acid, nitric acid, carbonic acid, phosphoric acid, and combinations thereof. Each possibility represents a separate embodiment.

According to one embodiment, the aqueous medium comprising a lactate salt of the present processes is derived from decomposed organic waste. According to another embodiment, the decomposed organic waste is obtained from a lactic acid fermentation.

According to some embodiments, the organic waste comprises a carbohydrate source. According to further embodiments, the organic waste is selected from the group consisting of food waste, municipal food waste, residential food waste, agricultural waste, industrial food waste from food processing facilities, commercial food waste (from hospitals, restaurants, shopping centers, airports etc.), and a mixture or combination thereof. Each possibility represents a separate embodiment.

Food waste in accordance with the present invention encompasses food waste and beverages of plant origin and/or animal origin. Food waste according to the present invention is typically mixed food waste, comprising one or more of bakery waste, dairy waste, animal-origin food waste including meat, poultry and fish waste, fruit and vegetable waste, and grain-based food waste (e.g., rice, couscous, pasta, noodles). Each possibility represents a separate embodiment. In some embodiments, mixed food waste according to the present invention comprises a combination of food wastes selected from bakery waste, dairy waste, animal-origin food waste including meat, poultry and fish waste, fruit and vegetable waste, and grain-based food waste (e.g., rice, couscous, pasta, noodles). Each possibility represents a separate embodiment. Food waste typically comprises solid components originating from food products or residues, e.g., food particles and debris, bones and bone fragments, shells and shell fragments, seeds and seed fragments, peels and the like, and also solids that do not originate from food products or residues, e.g., plastics, glass and metals, originating, for example, from packaging material.

According to some embodiments, the substrate for the lactic acid fermentation is a slurry of organic waste, particularly a slurry of food waste, or a liquid phase separated from a slurry of organic waste or pretreated slurry of organic waste. Each possibility represents a separate embodiment. An organic waste slurry (e.g., a food waste slurry) is typically characterized by a solid content (dry matter content) in the range of 5-50%, including each value within the specified range. In some embodiments, an organic waste slurry is characterized by a solid content in the range of 10-30%, including each value within the specified range. In other embodiments, an organic waste slurry is characterized by a solid content in the range of 15-35%, including each value within the specified range.

According to additional embodiments, an organic waste slurry is characterized by a water content in the range of 50-95%, including each value within the specified range. In some embodiments, an organic waste slurry is characterized by a water content in the range of 70%-90%, including each value within the specified range. In other embodiments, an organic waste slurry is characterized by a water content in the range of 65%-85%, including each value within the specified range.

According to various embodiments, the decomposed organic waste is obtained from a lactic acid-containing waste. According to other embodiments, the decomposed organic waste is obtained from hydrolysis of polylactic acid polymer.

According to further embodiments, the decomposed organic waste comprises lactic acid by-product that is derived from an industrial production of polylactic acid. In other embodiments, the decomposed organic waste comprises lactic acid by-product that is derived from industrial purification of lactic acid. In yet other embodiments, the decomposed organic waste comprises lactic acid obtained from hydrolyzing lactide by-product from an industrial production of polylactic acid.

According to various embodiments, the recovery of any one of the salt residues of the present processes can be performed using at least one of filtration, centrifugation, flotation, sedimentation, coagulation, flocculation, and decantation. Each possibility represents a separate embodiment. In one embodiment, the salt residues of the present processes are recovered by filtration (e.g., microfiltration) and/or centrifugation and washed with a suitable solvent prior to being reused in the processes.

It is to be understood that any combination of each of the aspects and the embodiments disclosed herein is explicitly encompassed within the disclosure of the present invention.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows a schematic representation of a process according to certain embodiments of the present invention.

FIG. 2 shows a schematic representation of a process according to certain embodiments of the present invention.

FIG. 3 shows a schematic representation of a process according to certain embodiments of the present invention.

FIG. 4 shows a schematic representation of a process according to certain embodiments of the present invention.

FIG. 5 shows a schematic representation of an electro-electrodialysis cell according to certain embodiments of the present invention.

### DETAILED DESCRIPTION OF THE INVENTION

The present invention provides processes for the production of lactic acid from a lactate salt solution or dispersion further comprising monovalent or divalent cations. The processes disclosed herein for the first time provide lactic acid production with minimal to no by-products or residues as the by-products or residues are recovered and reused, for example by recycling them back to different steps of the processes. In this manner, eco-friendly recycling processes are provided, which processes are also cost-effective as the reagents utilized therein can be obtained from previous productions. Furthermore, the processes of the present invention provide the direct production of lactic acid in high purity and yield.

For a better understanding of the invention and to show how it may be carried into effect, reference will now be made, purely by way of example, to the accompanying drawings. With specific reference now to the drawings in detail, it is stressed that the particulars shown are by way of example and for purposes of illustrative discussion of the preferred embodiments of the present invention only, and are presented with the purpose of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice.

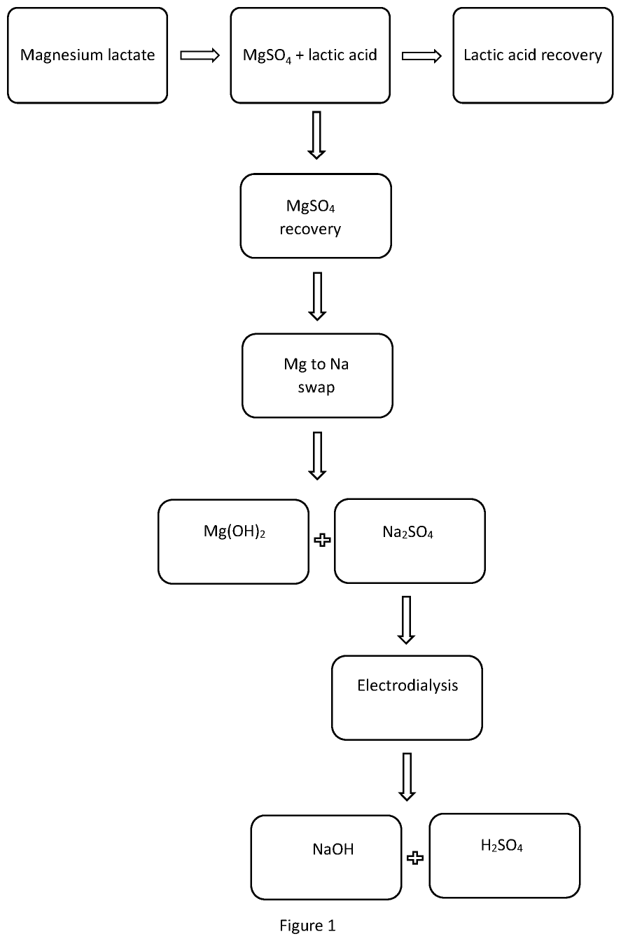

Reference is now made to FIG. 1 demonstrating certain features according to embodiments of the present invention. Lactic acid production is performed from an aqueous medium containing magnesium lactate. In some embodiments, the aqueous medium containing magnesium lactate is derived from decomposed organic waste. Typically, organic waste recycling is performed via fermentation of organic waste from municipal waste, food waste and agricultural waste utilizing L-lactic acid-producing microorganisms e.g., Bacillus coagulans. Due to the formation of L-lactic acid, endogenous lowering of the pH occurs. This endogenous lowering of the pH adversely affects the performance of the microorganisms. In order to avoid reaching a pH in which production of lactic acid is no longer feasible, the fermentation process is typically carried out in the presence of an alkaline substance to adjust the pH during fermentation. The alkaline substance neutralizes the pH resulting in the formation of L-lactate ions and counterions. In the setup outlined in FIG. 1, the alkaline substance utilized is magnesium hydroxide resulting in a fermentation broth comprising magnesium lactate.

When the magnesium lactate is acidified to lactic acid, using e.g., sulfuric acid, magnesium sulfate is obtained as a by-product. While the main stream of lactic acid production continues to downstream processing of lactic acid purification and separation, the magnesium sulfate by-product is subjected to a side sub-process which affords its recycling back to the process. In particular, the magnesium sulfate is subjected to a step of ion exchange or swap of the magnesium ions with sodium ions using a strong base (NaOH) to result in Mg(OH)₂ and Na₂SO₄. The magnesium hydroxide can then be separated and used in a subsequent fermentation process to maintain the pH during fermentation at the appropriate range suitable for the proper operation of the microorganisms. This mode of operation is highly advantageous by utilizing the alkaline substance formed during one batch production of lactic acid as the source of alkaline substance in a subsequent production of lactic acid.

The scheme further demonstrates the subsequent recycling of the Na₂SO₄ by-product of the swap reaction using electrodialysis to result in the production of NaOH and sulfuric acid. While the NaOH can be used for a subsequent swap reaction in this side sub-process, the sulfuric acid can be utilized for the acidification of the main process of lactic acid formation by protonating the lactate salt.

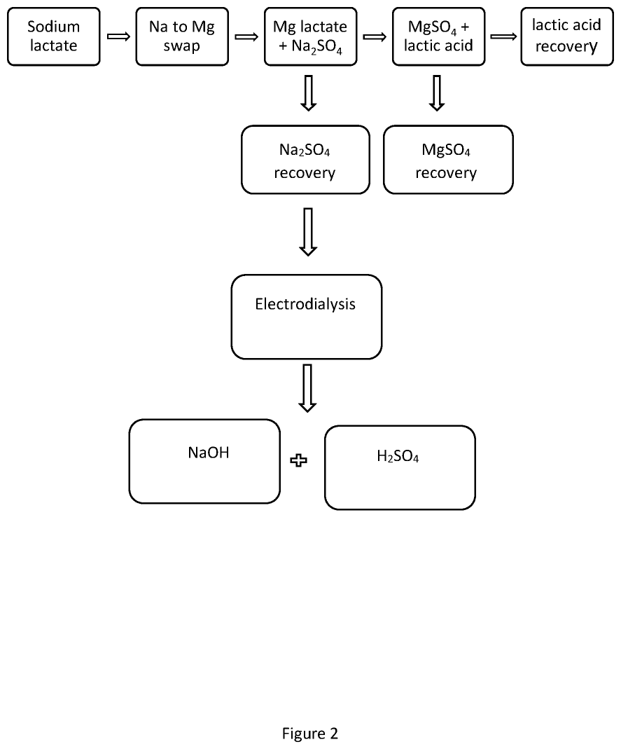

Reference is now made to FIG. 2 demonstrating certain features according to embodiments of the present invention. Lactic acid production is performed from an aqueous medium containing sodium lactate. The aqueous medium can be obtained, for example, from a fermentation broth in which NaOH is used as the alkaline substance to adjust the pH. The sodium lactate is subjected to a step of ion exchange or swap of the sodium ions with magnesium ions using a salt, for example MgSO₄, to produce magnesium lactate and Na₂SO₄. The magnesium lactate is then acidified to lactic acid, using e.g., sulfuric acid, and magnesium sulfate is obtained as a by-product. While the main stream of lactic acid production continues to downstream processing of lactic acid purification and separation, the magnesium sulfate by-product is recovered and reused for a subsequent ion swap of the sodium ions with magnesium ions. Furthermore, the Na₂SO₄ obtained as a by-product of the ion swap is recycled using electrodialysis to result in the production of NaOH and sulfuric acid, the sulfuric acid can be utilized for the acidification of the main process of lactic acid formation by protonating the lactate salt. The NaOH can be used as the alkaline substance to adjust the pH during fermentation to obtain an aqueous medium containing sodium lactate.

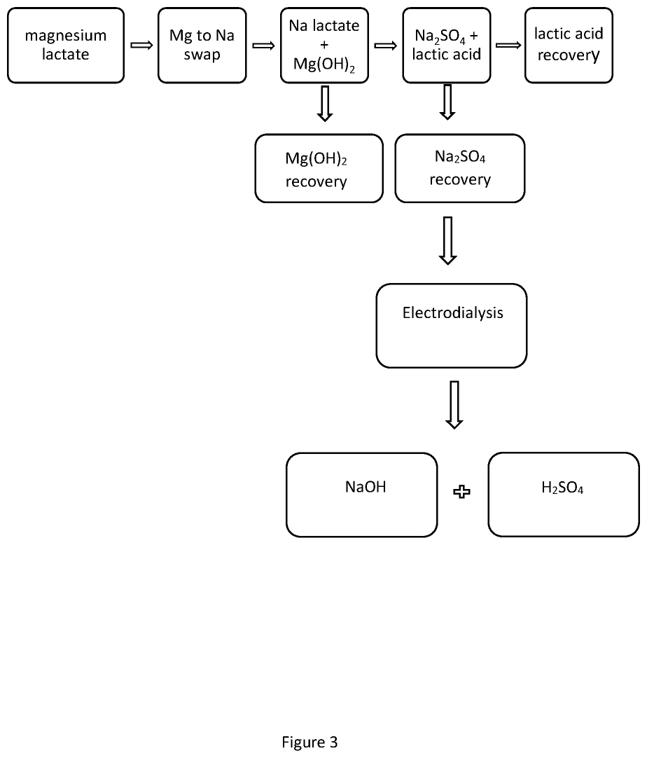

Reference is now made to FIG. 3 demonstrating certain features according to embodiments of the present invention. Lactic acid production is performed from an aqueous medium containing magnesium lactate which may be derived, for example, from lactic acid fermentation using magnesium hydroxide as an alkaline pH-adjusting agent as detailed above. The magnesium lactate is subjected to a step of ion exchange or swap of the magnesium ions with sodium ions using a first base, for example NaOH, to produce sodium lactate and magnesium hydroxide as the second base. The sodium lactate is then acidified to lactic acid, using e.g., sulfuric acid, and sodium sulfate is obtained as a by-product. While the main stream of lactic acid production continues to downstream processing of lactic acid purification and separation, the sodium sulfate by-product is recovered and recycled using electrodialysis to result in the production of NaOH and sulfuric acid, the sulfuric acid can be utilized for the acidulation of the main process of lactic acid formation by protonating the lactate salt. The NaOH can be used as the alkaline substance for the magnesium to sodium swap reaction.

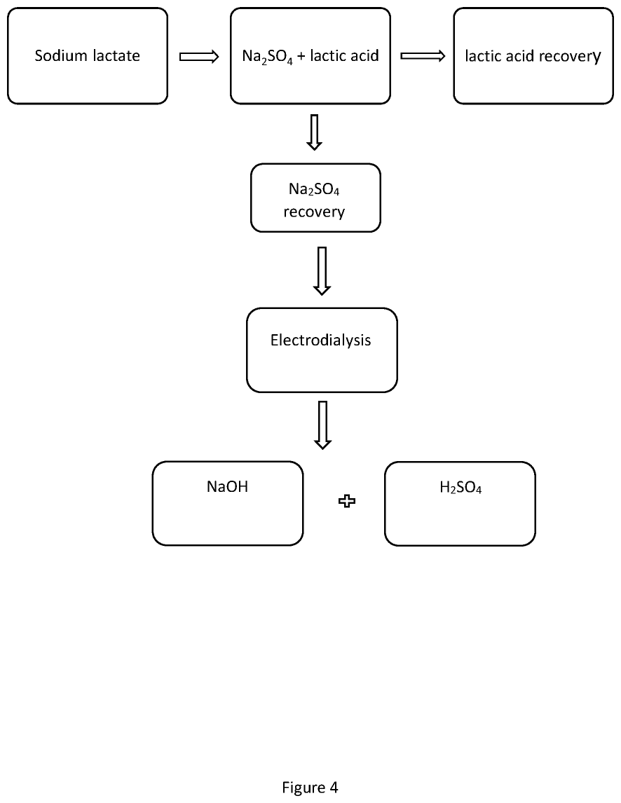

Reference is now made to FIG. 4 demonstrating certain features according to embodiments of the present invention. Lactic acid production is performed from an aqueous medium containing sodium lactate which may be derived, for example, from lactic acid fermentation using sodium hydroxide as an alkaline pH-adjusting agent as detailed above. The sodium lactate is acidified to lactic acid, using e.g., sulfuric acid, and sodium sulfate is obtained as a by-product. While the main stream of lactic acid production continues to downstream processing of lactic acid purification and separation, the sodium sulfate by-product is recovered and recycled using electrodialysis to result in the production of NaOH and sulfuric acid, the sulfuric acid can be utilized for the acidulation of the main process of lactic acid formation by protonating the lactate salt. The NaOH can be used as the alkaline substance to adjust the pH during fermentation to obtain an aqueous medium containing sodium lactate.

According to the principles disclosed herein, substantially all residues and by-products are recovered and reused in the processes of the present invention. The processes disclosed herein are therefore environmentally highly advantageous in avoiding residues that need to be discarded. The processes of the present invention also afford cost savings as expensive reagents are recycled and reused. Thus, commercial production of lactic acid in high purity and yield is provided, the lactic acid being particularly suitable for the preparation of polylactic acid.

According to some aspects and embodiments, an aqueous medium comprising lactate ions and divalent or monovalent counterions is obtained. According to various aspects and embodiments, the aqueous medium may be derived from decomposed organic waste or from a sugar fermentation process that produces a fermentation broth comprising lactate ions and divalent or monovalent counterions.

A fermentation broth containing lactate ions for use according to the present invention may be derived from lactic acid fermentation of various carbohydrate sources, including starch-based and cellulose-based carbohydrate sources. Examples of carbohydrate sources for fermentation include starches such as corn, potato and cassava starches, and cane sugar. Each possibility represents a separate embodiment. According to some embodiments, the carbohydrate source is organic waste, including starch-rich organic waste, lignocellulose-rich waste and dairy waste. Exemplary organic waste sources are detailed below.

According to certain embodiments, the aqueous medium is a decomposition product of any lactic acid-containing waste such as, but not limited to, polylactic acid polymer which was subjected to hydrolysis. According to other embodiments, a fermentation broth derived from organic waste feedstocks is used. Organic waste feedstocks within the scope of the present invention can be obtained from any waste source including, but not limited to, food waste, municipal food waste, residential food waste, agricultural waste, industrial food waste from food processing facilities, commercial food waste (from hospitals, restaurants, shopping centers, airports etc.), and a mixture or combination thereof. Each possibility represents a separate embodiment. The organic waste can additionally originate from residues ranging from animal and human excreta, vegetable and fruit residues, plants, cooked food, protein residues, slaughter waste, and combinations thereof. Each possibility represents a separate embodiment. Industrial organic food waste may include factory waste such as by products, factory rejects, market returns or trimmings of inedible food portions (such as skin, fat, crusts and peels). Each possibility represents a separate embodiment. Commercial organic food waste may include waste from shopping malls, restaurants, supermarkets, etc. Each possibility represents a separate embodiment.

According to various aspects and embodiments, the aqueous medium is a fermentation broth obtained from a fermentation process of a carbohydrate source. When using non-homogenous feedstocks, the fermentation broth typically comprises insoluble organic-based impurities such as, but not limited to, microorganisms (e.g., lactic acid producing microorganisms including e.g., yeasts, bacteria and fungi), fats and oils, lipids, aggregated proteins, bone fragments, hair, precipitated salts, cell debris, fibers (e.g., fruit and/or vegetables peels), and residual unprocessed waste (e.g., food shells, seeds, food insoluble particles and debris, etc.). Each possibility represents a separate embodiment. Non-limiting examples of insoluble inorganic-based impurities include plastics, glass, residues from food packaging, sand, and combinations thereof. Each possibility represents a separate embodiment. According to some embodiments, the insoluble impurities are removed from the fermentation broth. Removal of insoluble impurities can be achieved, for example, using at least one of filtration, centrifugation, flotation, sedimentation, coagulation, flocculation, and decantation. Each possibility represents a separate embodiment. Typically, removal is performed using filtration (e.g., microfiltration) and/or centrifugation.

Non-limiting examples of soluble impurities include water, solvents, polysaccharides, starch, cellulose, hemicellulose, lignin, seed fragment, salts, color components (e.g., tannins, flavonoids and carotenoids), and combinations thereof. Each possibility represents a separate embodiment. Typically, the soluble and insoluble impurities content of the broth is identical to the soluble and insoluble impurities content of the organic waste feedstocks. In some embodiments, the soluble and insoluble impurities content of the broth is lower by at least about 1 wt. % compared to the soluble and insoluble impurities content of the organic waste feedstocks. In further embodiments, the soluble and insoluble impurities content of the broth is lower by at least about 5 wt. %, about 10 wt. %, about 15 wt. %, about 20 wt. %, about 30 wt. %, about 40 wt. %, or about 50 wt. % compared to the soluble and insoluble impurities content of the organic waste feedstocks. Each possibility represents a separate embodiment.

According to some embodiments, the non-homogenous feedstock for the lactic acid fermentation is a slurry of organic waste, particularly food waste, or a liquid phase separated from a slurry of organic waste or pretreated slurry of organic waste (e.g., a slurry of organic waste that was subjected to saccharification and sterilization). As used herein, the term “slurry” of organic waste refers to a mixture of the organic waste and water, typically containing solid particles of the organic waste. A slurry of organic waste is typically formed by collecting waste material from various sources, subjecting the waste material to separation of plastics and inorganic solid components such as glass, metal and sand (to remove most and preferably all of the plastics and inorganic solid components), reducing the particle size of the waste material, e.g., by shredding or grinding, adding water if necessary, and forming a suspension of organic waste material in the water. In some embodiments, forming a slurry of organic waste, particularly a slurry of food waste, comprises subjecting the waste to depackaging, namely, removal of packaging material, including plastic, metal and glass packaging material. In some embodiments, the organic waste may naturally be in the form of a slurry.

An organic waste slurry (e.g., a food waste slurry) is typically characterized by a solid content (dry matter content) in the range of 5-50% (namely, characterized by a liquid or moisture content in the range of 50-95%), including each value within the specified ranges. In some embodiments, an organic waste slurry is characterized by a solid content in the range of 10-30% (namely, a liquid or moisture content in the range of 70%-90%), including each value within the specified ranges. In some other embodiments, an organic waste slurry is characterized by a solid content in the range of 15-35% (namely, a liquid or moisture content in the range of 65%-85%), including each value within the specified ranges.

According to further embodiments, the aqueous medium may be a by-product of industrial production of polylactic acid or industrial purification of lactic acid. According to yet other embodiments, the aqueous medium is a by-product of hydrolysis of lactide from an industrial production of polylactic acid.

According to the principles of the present invention, the aqueous medium comprises lactate ions at concentrations of about 10 g/L to about 500 g/L, including each value within the specified range. Exemplary lactate concentrations include, but are not limited to, about 10 g/L, about 25 g/L, about 50 g/L, about 75 g/L, about 100 g/L, about 125 g/L, about 150 g/L, about 175 g/L, about 200 g/L, about 225 g/L, about 250 g/L, about 275 g/L, about 300 g/L, about 325 g/L, about 350 g/L, about 375 g/L, about 400 g/L, about 425 g/L, about 450 g/L, about 475 g/L, or about 500 g/L. Each possibility represents a separate embodiment.

When the lactate counterions are divalent ions (e.g., magnesium ions or calcium ions), the lactate salt is acidified or protonated by adding an acid thereby resulting in the formation of lactic acid and a divalent salt residue. When the lactate counterions are monovalent ions (e.g., sodium ions or ammonium ions), the lactate salt is acidified or protonated by adding an acid thereby resulting in the formation of lactic acid and a monovalent salt residue. The term “lactic acid” as used herein refers to the hydroxycarboxylic acid having the following chemical formula CH₃CH(OH)CO₂H. The terms lactic acid or lactate (unprotonated lactic acid) can refer to the stereoisomers (enantiomers) of lactic acid including L-lactic acid/L-lactate, D-lactic acid/D-lactate, or to a combination thereof. Each possibility represents a separate embodiment.

Any acid can be utilized in acidulation of lactate salt including, but not limited to, organic or inorganic acids. Such acids include hydrochloric, hydrofluoric, trifluoroacetic, sulfuric, phosphoric, acetic, succinic, citric, lactic, maleic, fumaric, palmitic, cholic, pamoic, mucic, D-glutamic, D-camphoric, glutaric, phthalic, tartaric, lauric, stearic, salicylic, methanesulfonic, benzenesulfonic, sorbic, picric, benzoic, cinnamic, and the like. Each possibility represents a separate embodiment. Exemplary acids include, but are not limited to, sulfuric acid, hydrochloric acid, hydrobromic acid, nitric acid, carbonic acid, phosphoric acid, and combinations thereof. Each possibility represents a separate embodiment. Currently preferred is the use of sulfuric acid.

According to some aspects and embodiments, the addition of an acid is performed at elevated temperatures, for example at about 40° C. to about 80° C., preferably about 50° C. to about 70° C., including each value within the specified ranges. Exemplary temperatures include, but are not limited to, about 40° C., about 45° C., about 50° C., about 55° C., about 60° C., about 65° C., about 70° C., about 75° C., and about 80° C. Each possibility represents a separate embodiment. Due to the exothermic reaction upon acid addition, the addition can be performed while cooling, for example using a cooling jacket to maintain the temperatures at the desired range. The rate of addition may vary but typically includes a slow rate to maintain the temperature at the desired range and avoid overheating. Suitable rates at which acid can be added include, but are not limited to, about 0.5% to about 5% per minute, for example about 1% to about 4% per minute, including each value within the specified ranges. Exemplary rates include, but are not limited to, about 0.5%/min, about 1%/min, about 1.5%/min, about 2%/min, about 2.5%/min, about 3%/min, about 3.5%/min, about 4%/min, about 4.5%/min, and about 5%/min. Each possibility represents a separate embodiment.

Within the scope of the present invention is the addition of an acid in a continuous manner by continuously feeding the lactate salt with essentially equimolar rates of acid corresponding to the lactate to be acidified.

Following acid addition, the acidulate is typically maintained at the elevated temperatures while mixing followed by cooling to room temperatures. Thereafter, an organic solvent is added to precipitate the salt and divalent or monovalent cation residues. Suitable organic solvents within the scope of the present invention include, but are not limited to, ketones, aldehydes, ethers, esters, nitriles, amides, halogenated hydrocarbons, aromatic hydrocarbons, and mixtures or combinations thereof. Each possibility represents a separate embodiment. Exemplary organic solvents include, but are not limited to, ketones, ethers, aldehydes, and a mixture thereof. Each possibility represents a separate embodiment. Currently preferred is the use of ketones, particularly acetone or ethyl acetate. The organic solvent being added is typically maintained at lower temperatures in order to avoid solvent evaporation. Suitable temperatures of the organic solvent include 25° C. or less, for example about 5° C. to about 25° C., preferably about 5° C. to about 15° C., including each value within the specified ranges. Exemplary temperatures of the organic solvent include, but are not limited to, about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., and about 25° C. Each possibility represents a separate embodiment.

The organic solvent can be added to the mixture of lactic acid and residual divalent or monovalent salt at a ratio of 1:10 to 10:1, including all iterations of ratios within the specified range. Exemplary ratios include, but are not limited to, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. Each possibility represents a separate embodiment. Typically, the residual divalent or monovalent salt is precipitated and separated from the slurry for use in the side sub-process. The remaining lactic acid-containing mixture is then further processed to collect the lactic acid as is known in the art.

According to the principles of the present invention, where a divalent salt residue is produced, it is then treated to be recycled back to the process as a reagent for a subsequent swap reaction. In some embodiments, the divalent salt residue includes, but is not limited to, MgCl₂·xH₂O where x=0-6; MgCO₃·xH₂O where x=0, 1, 2, 3, or 5; MgSO₄·xH₂O where x=0-11; Mg₃(PO₄)₂·xH₂O where x=0, 5, 8, or 22; MgHPO₄·xH₂O where x=0 or 3; and Mg(H₂PO₄)₂·xH₂O where x=0, 2 or 4. Each possibility represents a separate embodiment. Currently preferred divalent salt residue is MgSO₄·7H₂O.

In embodiments where monovalent salt residues are formed, they can be collected for subsequent reuse (e.g., ammonium sulfate salt may be isolated and used in agriculture) or subjected to further processing (e.g., electrodialysis) as detailed below.

According to some aspects and embodiments, ion swap between divalent cations and monovalent cations or vice versa is performed. Typically, the ion swap is performed by the addition of a monovalent base to the divalent salt residue or to the divalent lactate salt. Suitable monovalent bases within the scope of the present invention include, but are not limited to, sodium hydroxide, potassium hydroxide, ammonium hydroxide, and a mixture thereof. Each possibility represents a separate embodiment. Currently preferred is the addition of sodium hydroxide or ammonium hydroxide. Within the scope of the present invention is the use of ammonia water (aqua ammonia) as the base comprising the monovalent ions.

According to some aspects and embodiments, the addition of a monovalent base is performed at temperatures of about 20° C. to about 80° C., preferably about 30° C. to about 60° C., including each value within the specified ranges. Exemplary temperatures include, but are not limited to, about 20° C., about 25° C., about 30° C., about 35° C., about 40° C., about 45° C., about 50° C., about 55° C., about 60° C., about 65° C., about 70° C., about 75° C., and about 80° C. Each possibility represents a separate embodiment. When sodium hydroxide is added as the monovalent base to a magnesium sulfate salt residue, sodium sulfate salt is formed. Due to the unique solubility curve of Na₂SO₄, it is recommended to add the sodium hydroxide monovalent base at a temperature in the range of 35-50° C. in order to avoid precipitation of Na₂SO₄ with the Mg(OH)₂. However, it is to be understood that lower temperatures can be utilized at lower concentrations.

While the monovalent base can be added in stochiometric amounts, the present invention also contemplates the addition of the monovalent base in excess. Up to 20% excess of the monovalent base can be added according to the principles of the present invention. Typically, the addition of monovalent base is performed gradually e.g., dropwise. Suitable rates at which the monovalent base can be added include, but are not limited to, about 0.01% to about 5% per minute, for example about 0.5% to about 2.5% per minute, including each value within the specified ranges. Exemplary rates include, but are not limited to, about 0.01%/min, about 0.05%/min, about 0.1%/min, about 0.5%/min, about 1%/min, about 1.5%/min, about 2%/min, about 2.5%/min, about 3%/min, about 3.5%/min, about 4%/min, about 4.5%/min, and about 5%/min. Each possibility represents a separate embodiment.

Within the scope of the present invention is the addition of a base in a continuous manner by continuously feeding the divalent salt residue or the lactate salt with the base.

Where a divalent base is formed after ion swap, the divalent base includes, but is not limited to, magnesium hydroxide or calcium hydroxide with each possibility representing a separate embodiment. According to the principles of the present invention the divalent base thus obtained is recycled back to the process to adjust the pH in the lactic acid fermentation or for hydrolyzing polylactic acid.

According to other aspects and embodiments, ion swap is performed by the addition of a divalent salt to the monovalent lactate. Suitable divalent salts within the scope of the present invention include, but are not limited to, MgCl₂·xH₂O where x=0-6; MgCO₃·xH₂O where x=0, 1, 2, 3, or 5; MgSO₄·xH₂O where x=0-11; Mg₃(PO₄)₂·xH₂O where x=0, 5, 8, or 22; MgHPO₄·xH₂O where x=0 or 3; and Mg(H₂PO₄)₂·xH₂O where x=0, 2 or 4. Each possibility represents a separate embodiment. Currently preferred is the addition of MgSO₄·7H₂O.

According to some aspects and embodiments, the addition of a divalent salt to the monovalent lactate is performed at temperatures of about 20° C. to about 80° C., including each value within the specified range. Exemplary temperatures include, but are not limited to, about 20° C., about 25° C., about 30° C., about 35° C., about 40° C., about 45° C., about 50° C., about 55° C., about 60° C., about 65° C., about 70° C., about 75° C., and about 80° C. Each possibility represents a separate embodiment.

While the divalent salt can be added in stochiometric amounts, the present invention also contemplates the addition of the divalent salt in excess. Up to 20% excess of the divalent salt can be added according to the principles of the present invention.

Following acidulation and/or swap reactions, salt residues comprising monovalent cations are formed. Within the scope of the present invention is the formation of salt residues that include, but are not limited to, sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof. Each possibility represents a separate embodiment. Currently preferred is the formation of sodium sulfate or ammonium sulfate. Each possibility represents a separate embodiment.

According to the principles of the present invention, the thus obtained monovalent salt by-products are collected for subsequent reuse. For example, ammonium sulfate salt may be isolated and used in agriculture.

According to certain aspects and embodiments, the thus obtained monovalent salts by-products are subjected to electrodialysis.

Electrodialysis apparatuses are typically composed of two electrodes, an anode and a cathode, separated from one another by one or more ion-selective membranes. Ion-selective membranes suitable for use in the present invention include, but are not limited to, anion-selective, cation-selective and bipolar membranes. Each possibility represents a separate embodiment.

In some embodiments, the aqueous medium comprising the monovalent salt by-products is introduced into the feed chamber and the monovalent cations are transported from the feed chamber to the base chamber through the cation exchange membrane to produce a base solution comprising the monovalent cations. Simultaneously, anions are transported through the anion exchange membrane to the acid chamber to produce the second solution comprising the acid. In other embodiments, the aqueous medium comprising the monovalent salt by-product is introduced into the feed chamber where the acid is formed and the monovalent cations are transported from the feed chamber to the base chamber through the cation exchange membrane to produce a base solution comprising the monovalent cations. In this manner, the acid and the salt solution (i.e., brine) are mixed in the feed chamber. In accordance with these embodiments, the acid may further be separated via distillation before its reused or alternatively reused as a mixture that further comprises the brine.

Within the scope of the present invention are electrodialysis modes of operation selected from the group consisting of water-splitting electrodialysis (WSED), electro-electrodialysis (EED), and bipolar membrane electrodialysis (BPED). Each possibility represents a separate embodiment. Currently preferred is the use of BPED whereby a bipolar membrane is utilized to convert a saline solution comprising the monovalent salt into the corresponding acid and base under the influence of an electrical field.

Typically, the flow rate at which the monovalent salt by-product is introduced into the feed chamber is between about 1 dm³/h to about 1000 dm³/h, for example about 10 dm³/h to about 500 dm³/h, including each value within the specified ranges. Exemplary flow rates include, but are not limited to, about 1 dm³/h, about 5 dm³/h, about 10 dm³/h, about 20 dm³/h, about 30 dm³/h, about 40 dm³/h, about 50 dm³/h, about 60 dm³/h, about 70 dm³/h, about 80 dm³/h, about 90 dm³/h, about 100 dm³/h, about 200 dm³/h, about 300 dm³/h, about 400 dm³/h, about 500 dm³/h, about 600 dm³/h, about 700 dm³/h, about 800 dm³/h, about 900 dm³/h, and about 1000 dm³/h. Each possibility represents a separate embodiment.

The concentration of the monovalent salt fed into the electrodialysis is typically in the range of about 10 g/L to about 200 g/L, for example about 50 g/L to about 200 g/L, including each value within the specified ranges. Exemplary concentrations include, but are not limited to, about 10 g/L, about 20 g/L, about 30 g/L, about 40 g/L, about 50 g/L, about 60 g/L, about 70 g/L, about 80 g/L, about 90 g/L, about 100 g/L, about 110 g/L, about 120 g/L, about 130 g/L, about 140 g/L, about 150 g/L, about 160 g/L, about 170 g/L, about 180 g/L, about 190 g/L, and about 200 g/L. Each possibility represents a separate embodiment.

While electrodialysis can be performed at room temperatures, it can also be performed at elevated temperatures. Suitable temperatures at which electrodialysis can be performed include from about 20° C. to about 80° C., for example from about 40° C. to about 70° C., including each value within the specified ranges. Exemplary temperatures for performing the electrodialysis include, but are not limited to, about 20° C., about 25° C., about 30° C., about 35° C., about 40° C., about 45° C., about 50° C., about 55° C., about 60° C., about 65° C., about 70° C., about 75° C., and about 80° C. Each possibility represents a separate embodiment.

According to the principles of the present invention the products of electrodialysis are an acid and a base comprising monovalent cations. Exemplary bases comprising monovalent cations include, but are not limited to sodium hydroxide, potassium hydroxide, and ammonium hydroxide. Each possibility represents a separate embodiment. Exemplary acids include, but are not limited to, sulfuric acid, hydrochloric acid, hydrobromic acid, nitric acid, carbonic acid, phosphoric acid, and combinations thereof. Each possibility represents a separate embodiment.

According to the principles of the present invention, the products of the electrodialysis are recycled back to the process whereby the acid is reused in the step of acidifying the lactate salt to lactic acid, and the monovalent base is reused in the step of ion-exchange or swap or alternatively as an alkaline substance for obtaining an aqueous medium comprising lactate ions such as, for example, for adjusting the pH in a lactic acid fermentation or for hydrolyzing polylactic acid.

Within the scope of the present invention is the recovery of any one of the solid intermediate products using at least one of filtration, centrifugation, flotation, sedimentation, coagulation, flocculation, and decantation. Each possibility represents a separate embodiment. Currently preferred is the recovery by filtration (e.g., microfiltration and/or nanofiltration) and/or centrifugation. The obtained intermediate products are separated from the remaining liquid and may be washed with an aqueous solution or with an organic solvent such as ethanol or acetone and purified prior to being reused in the process. Further encompassed within the scope of the present invention is the concentration of liquid intermediate products such as the acid and base obtained following electrodialysis to desirable acid and base concentrations. It is to be understood that any one of the residues or by-products that are recovered and reused according to the principles of the present invention may be further purified by e.g., distillation, solid-liquid separation, crystallization, and the like, prior to being reused.

The term “about” as used herein refers to ±10% of a specified value.

Throughout the description and claims, the words “comprise” and “contain” and variations of the words, for example “comprising” and “comprises”, mean “including but not limited to”, and are not intended to (and do not) exclude other moieties, additives, components, integers or steps.

As used herein, the singular forms “a”, “an”, and “the” include plural forms unless the context clearly dictates otherwise. Thus, for example, reference to “a cation” includes combinations of cations as is known in the art.

As used herein, the term “and” or the term “or” include “and/or” unless the context clearly dictates otherwise.

The following examples are presented in order to more fully illustrate some embodiments of the invention. They should, in no way be construed, however, as limiting the broad scope of the invention. One skilled in the art can readily devise many variations and modifications of the principles disclosed herein without departing from the scope of the invention.

### EXAMPLES

### Example 1. Acidulation of Magnesium Lactate

In a 3 L glass reactor equipped with a mechanical stirrer and a thermometer, 571 g of MgLa₂ crystals were dissolved in 387 g of DW to a 45% dry matter slurry. The slurry was heated to 60° C. and stirred at 300 RPM. 172 g of H₂SO₄ 96% were added using a dropping funnel over the course of 1 hour. After the addition, the acidulate was stirred for 1 hour under the same conditions, then cooled to 25° C. 1135 g of acetone at 20° C. were added to the reactor, followed by immediate precipitation of MgSO₄. The mixture was stirred vigorously for 2 hours, followed by a vacuum filtration using a P3 sintered glass funnel, removing the MgSO₄. The MgSO₄ cake was washed with additional 340 g of acetone, the wash was combined with the original filtrate. The acetone was evaporated and recovered using a rotary evaporator at 40° C. and 150 mbar. 671 g of 48% w/w lactic acid were obtained. Yield: 97%.

### Example 2. Divalent to Monovalent Cation Exchange

The MgSO₄ solution of Example 1 is subjected to an ion swap by the addition of NaOH. In particular, MgSO₄ is mixed with water to a 50% w/w slurry, and the mixture is heated to about 40° C. 2 molar equivalents of NaOH 10M solution are added dropwise, resulting in precipitation of Mg(OH)₂ as a white solid. The precipitated solid is separated via filtration or centrifugation and washed with water. The Mg(OH)₂ is recycled and reused as a base in subsequent lactic acid fermentation. The solution containing Na₂SO₄ is subjected to electrodialysis as described in Example 6.

### Example 3. Monovalent to Divalent Cation Exchange—Sodium Lactate

A sodium lactate solution is subjected to an ion swap by the addition of magnesium sulfate. In particular, the swap reaction is performed at 20-80° C. with 0-20% excess of MgSO₄·xH₂O (x=0-11) which is added to the mixed solution. The precipitated solid is separated via filtration or centrifugation and washed with water to typically afford crude MgLa₂·2H₂O which is subjected to acidulation as described in Example 1 and the magnesium sulfate obtained as a byproduct of acidulation is recycled and reused in a subsequent sodium to magnesium swap reaction. The solution containing Na₂SO₄ is subjected to electrodialysis as described in Example 6.

### Example 4. Monovalent to Divalent Cation Exchange—Magnesium Lactate

Magnesium lactate was subjected to an ion swap to sodium lactate by mixing with a sodium hydroxide base. 100 g of NaOH 40% were added to a 0.5 L reactor equipped with temperature and pH probes, heated to 90° C. and stirred at 300 RPM. 20% MgLa₂ slurry was added using a peristaltic pump at 12 RPM, over the course of about 1 h. Following the addition of about 200 g of the MgLa₂ slurry, the reaction medium solidified completely. The stirring was increased to 600 RPM to mechanically break the solid. A total of 450 g of MgLa₂ were added. The solution was centrifuged at 12,000 g for 5 min to separate the precipitated MgOH₂ from the sodium lactate solution. The Mg(OH)₂ precipitate was washed with 2 weight equivalents of DW and the wash was combined with the filtrate. NaOH residues were neutralized with lactic acid. Lactate recovery yield: 97%. Magnesium recovery yield: 98%.

### Example 5. Acidulation of Sodium Lactate

In a glass reactor, equipped with a mechanical stirrer and a thermometer, sodium lactate solution obtained as described in Example 4 is heated to 60° C. and stirred at 300 RPM. 1 molar equivalent of H₂SO₄ 96% is added using a dropping funnel over the course of 1 hour. After the addition, the acidulate is stirred for 1 hour under the same conditions, then cooled to 25° C. 1 weight equivalent of acetone at 20° C. is added to the reactor, followed by immediate precipitation of Na₂SO₄. The mixture is stirred vigorously for 2 hours, followed by a vacuum filtration using a P3 sintered glass funnel, removing the Na₂SO₄. The Na₂SO₄ cake is washed with additional 0.3 weight equivalents of acetone and subjected to electrodialysis as described in Example 6. The wash containing the lactic acid is combined with the original filtrate followed by the evaporation of the acetone and recovery using a rotary evaporator at 40° C. and 150 mbar.

### Example 6. Electrodialysis

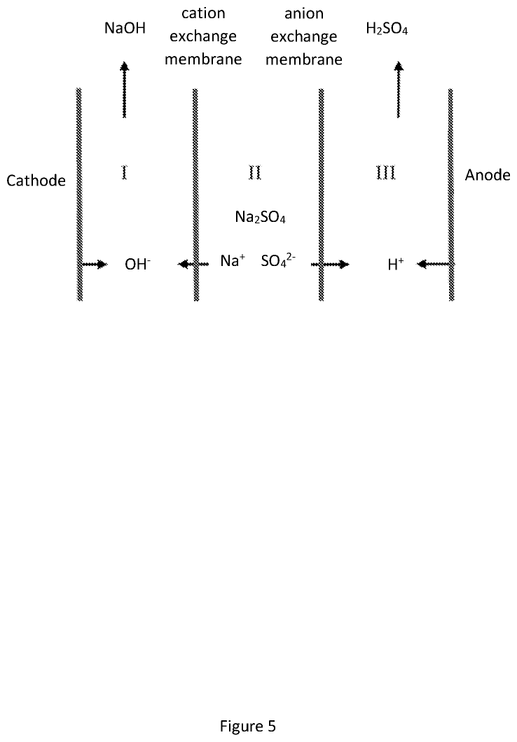

Electro-electrodialysis process is performed on the Na₂SO₄ solution of Examples 2, 3, or 5 to simultaneously produce H₂SO₄ and NaOH. A cell containing three chambers including an acid chamber, a feed chamber, and a base chamber is used (FIG. 5). The solution containing the Na₂SO₄ is fed to the feed chamber at an initial volume of 300 mL-800 mL. The flow rate is 40-80 dm³/h. Electrodialysis is performed at initial concentrations of Na₂SO₄ of 50-200 g/L.

The H₂SO₄ produced at the acid chamber is recycled back to acidify the lactate salt (Example 1) and the NaOH is recycled back to the ion swap (Example 2) or to the formation of a sodium lactate solution (Example 3). Alternatively, The H₂SO₄ produced at the acid chamber is recycled back to acidify the lactate salt (Example 4) and the NaOH is recycled back to the ion swap (Example 5) or to the subsequent formation of a sodium lactate solution (without swap).

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-48. (canceled)

49. A process for the preparation of lactic acid, the process comprising the steps of:
a. providing an aqueous medium comprising a lactate salt with divalent cations;
b. adding an acid to the aqueous medium of step (a) to obtain lactic acid and a salt residue comprising the divalent cations;
c. reacting the salt residue comprising the divalent cations of step (b) with a first base comprising monovalent cations to obtain a second base comprising the divalent cations and a salt residue comprising the monovalent cations; and
d. collecting the salt residue comprising the monovalent cations of step (c),
wherein the second base comprising the divalent cations of step (c) is recovered and reused in the process of producing an aqueous medium of step (a).

50. The process according to claim 49, wherein the divalent cations are magnesium ions.

51. The process according to claim 49, wherein the acid in step (b) is selected from the group consisting of sulfuric acid, hydrochloric acid, hydrobromic acid, nitric acid, carbonic acid, phosphoric acid, and combinations thereof, and/or wherein the salt residue comprising the divalent cations is selected from the group consisting of MgCl₂·xH₂O where x=0-6; MgCO₃·xH₂O where x=0, 1, 2, 3, or 5; MgSO₄·xH₂O where x=0-11; Mg₃(PO₄)₂·x H₂O where x=0, 5, 8, or 22; MgHPO₄·xH₂O where x=0 or 3; and Mg(H₂PO₄)₂·x H₂O where x=0, 2 or 4; and/or wherein the lactic acid obtained in step (b) is recovered using an organic solvent selected from the group consisting of a ketone, an ether, an aldehyde, and a mixture thereof.

52. The process according to claim 49, wherein the first base comprising monovalent cations in step (c) is selected from the group consisting of sodium hydroxide, potassium hydroxide, ammonium hydroxide, and a mixture thereof.

53. The process according to claim 49, wherein the salt residue comprising the monovalent cations is selected from sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof.

54. The process according to claim 49, further comprising step (e) of subjecting the collected salt residue comprising the monovalent cations of step (d) to electrodialysis thereby obtaining a base comprising the monovalent cations and an acid, wherein the base comprising the monovalent cations is recovered and reused as the first base in step (c), and/or the acid is recovered and reused in step (b).

55. The process according to claim 54, wherein the electrodialysis in step (e) is bipolar membrane electrodialysis (BPED); and/or wherein the base comprising the monovalent cations is selected from sodium hydroxide, potassium hydroxide, and ammonium hydroxide; and/or wherein the acid is selected from the group consisting of sulfuric acid, hydrochloric acid, hydrobromic acid, nitric acid, carbonic acid, phosphoric acid, and combinations thereof.

56. A process for the preparation of lactic acid, the process comprising the steps of:
a. providing an aqueous medium comprising a lactate salt with monovalent cations;
b. reacting the lactate salt with monovalent cations of step (a) with a first salt comprising divalent cations to obtain a lactate salt with divalent cations and a second salt residue comprising the monovalent cations;
c. adding an acid to the lactate salt with divalent cations of step (b) to obtain lactic acid and a salt residue comprising the divalent cations; and
d. collecting the second salt residue comprising the monovalent cations of step (b),
wherein the salt residue comprising the divalent cations of step (c) is recovered and reused as the first salt in step (b).

57. The process according to claim 56, wherein the monovalent cations are sodium ions.

58. The process according to claim 56, wherein the first salt comprising divalent cations is selected from the group consisting of MgCl₂·xH₂O where x=0-6; MgCO₃·xH₂O where x=0, 1, 2, 3, or 5; MgSO₄·x H₂O where x=0-11; Mg₃(PO₄)₂·x H₂O where x=0, 5, 8, or 22; MgHPO₄·x H₂O where x=0 or 3; and Mg(H₂PO₄)₂·x H₂O where x=0, 2 or 4; or wherein the second salt residue comprising the monovalent cations is selected from sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof.

59. The process according to claim 56, wherein the acid in step (c) is selected from the group consisting of sulfuric acid, hydrochloric acid, hydrobromic acid, nitric acid, carbonic acid, phosphoric acid, and combinations thereof, and/or wherein the lactic acid obtained in step (c) is recovered using an organic solvent selected from the group consisting of a ketone, an ether, an aldehyde, and a mixture thereof.

60. The process according to claim 56, further comprising step (e) of subjecting the collected second salt residue comprising the monovalent cations of step (d) to electrodialysis thereby obtaining a base comprising the monovalent cations and an acid, wherein the base comprising the monovalent cations is recovered and reused in the process of producing an aqueous medium of step (a), and/or the acid is recovered and reused in step (c).

61. The process according to claim 60, wherein the electrodialysis in step (e) is bipolar membrane electrodialysis (BPED); and/or wherein the acid is selected from the group consisting of sulfuric acid, hydrochloric acid, hydrobromic acid, nitric acid, carbonic acid, phosphoric acid, and combinations thereof, and/or wherein the base comprising the monovalent cations is selected from sodium hydroxide, potassium hydroxide, and ammonium hydroxide.

62. A process for the preparation of lactic acid, the process comprising the steps of:
a. providing an aqueous medium comprising a lactate salt with divalent cations;
b. reacting the lactate salt with divalent cations of step (a) with a first base comprising monovalent cations to obtain a second base comprising the divalent cations and a lactate salt comprising the monovalent cations;
c. adding an acid to the lactate salt comprising the monovalent cations of step (b) to obtain lactic acid and a salt residue comprising the monovalent cations; and
d. performing electrodialysis on the salt residue comprising the monovalent cations of step (c) to obtain a base comprising the monovalent cations and an acid,
wherein the second base comprising the divalent cations of step (b) is recovered and reused in the process of producing an aqueous medium of step (a), the base comprising the monovalent cations of step (d) is recovered and reused as the first base in step (b), and/or the acid in step (d) is recovered and reused in step (c).

63. The process according to claim 62, wherein the divalent cations are magnesium ions.

64. The process according to claim 62, wherein the first base comprising monovalent cations in step (b) is selected from the group consisting of sodium hydroxide, potassium hydroxide, ammonium hydroxide, and a mixture thereof; and/or wherein the acid in step (c) is selected from the group consisting of sulfuric acid, hydrochloric acid, hydrobromic acid, nitric acid, carbonic acid, phosphoric acid, and combinations thereof; and/or wherein the lactic acid obtained in step (c) is recovered using an organic solvent selected from the group consisting of a ketone, an ether, an aldehyde, and a mixture thereof; and/or wherein the salt residue comprising the monovalent cations is selected from the group consisting of sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof.

65. The process according to claim 62, wherein the electrodialysis in step (d) is bipolar membrane electrodialysis (BPED); and/or wherein the base comprising the monovalent cations in step (d) is selected from the group consisting of sodium hydroxide, potassium hydroxide, and ammonium hydroxide; and/or wherein the acid in step (d) is selected from the group consisting of sulfuric acid, hydrochloric acid, hydrobromic acid, nitric acid, carbonic acid, phosphoric acid, and combinations thereof.

66. A process for the preparation of lactic acid, the process comprising the steps of:
a. providing an aqueous medium comprising a lactate salt with monovalent cations;
b. adding an acid to the lactate salt with monovalent cations of step (a) to obtain lactic acid and a salt residue comprising the monovalent cations; and
c. performing electrodialysis on the salt residue comprising the monovalent cations of step (b) to obtain a base comprising the monovalent cations and an acid,
wherein the base comprising the monovalent cations of step (c) is recovered and reused in the process of producing an aqueous medium of step (a), and/or the acid in step (c) is recovered and reused in step (b).

67. The process according to claim 66, wherein the acid in step (b) is selected from the group consisting of sulfuric acid, hydrochloric acid, hydrobromic acid, nitric acid, carbonic acid, phosphoric acid, and combinations thereof, or wherein the lactic acid obtained in step (b) is recovered using an organic solvent selected from the group consisting of a ketone, an ether, an aldehyde, and a mixture thereof, or wherein the salt residue comprising the monovalent cations is selected from the group consisting of sodium sulfate, potassium sulfate, ammonium sulfate, sodium chloride, potassium chloride, ammonium chloride, sodium bromide, potassium bromide, ammonium bromide, sodium nitrate, potassium nitrate, ammonium nitrate, sodium carbonate, potassium carbonate, ammonium carbonate, sodium bicarbonate, potassium bicarbonate, ammonium bicarbonate, sodium phosphate, potassium phosphate, ammonium phosphate, and combinations thereof, and/or wherein the electrodialysis in step (c) is bipolar membrane electrodialysis (BPED); and/or wherein the base comprising the monovalent cations in step (c) is selected from the group consisting of sodium hydroxide, potassium hydroxide, and ammonium hydroxide; and/or wherein the acid in step (c) is selected from the group consisting of sulfuric acid, hydrochloric acid, hydrobromic acid, nitric acid, carbonic acid, phosphoric acid, and combinations thereof.

68. The process according to claim 49, wherein the aqueous medium in step (a) is derived from decomposed organic waste, wherein the decomposed organic waste is obtained from a lactic acid fermentation; and/or wherein the organic waste is selected from the group consisting of food waste, municipal food waste, residential food waste, agricultural waste, industrial food waste from food processing facilities, commercial food waste, and a mixture or combination thereof.
